(19) United States
(12) Patent Application Publication
(10) Pub. No.: US 2020/0054455 A1
(43) Pub. Date: Feb. 20, 2020

(54) INTERBODY STANDALONE DEVICE WITH INTEGRATED FIXATIONS

(71) Applicant: Mohsen Sharifi Renani, Isfahan (IR)

(72) Inventor: Mohsen Sharifi Renani, Isfahan (IR)

(21) Appl. No.: 16/514,998

(22) Filed: Jul. 17, 2019

Related U.S. Application Data
(60) Provisional application No. 62/715,279, filed on Aug. 7, 2018.

(51) Int. Cl. A61F 2/30; A61F 2/44

(52) U.S. Cl. CPC A61F 2/30749; A61F 2002/2835; A61F 2/447; A61F 2/442

(57) ABSTRACT

In the present invention, stand-alone interbody spine implant for fusion of adjacent vertebrae is disclosed. In the preferred configuration, this implant includes one spacer which is placed between adjacent vertebrae and at least one pin/bar/fastener which attaches the spacer to the bones. The spacer includes channel(s) with various configurations which guide the pins/bars/fasteners through the spacer in order to penetrate into bone. The combination of pins/bars/fasteners and spacer together, provides fixation of the implant to bone and fuses the adjacent vertebrae.

### CROSS REFERENCE TO RELATED APPLICATIONS

The present application claims priority to U.S. Provisional Patent Application entitled “Interbody Standalone Device With Integrated Fixations”, Ser. No. 62/715,279 filed on Aug. 7, 2018, which is incorporated by reference herein in its entirety.

### BACKGROUND

### 1. Field of the Invention

The present invention generally relates to methods and devices for the treatment of the spinal disorder.

The human spine is a complex bony structure in the back of the body, which is intended to support the weight of the upper body, provide posture while allowing for movement and flexibility, and protect the spinal cord. The spine is composed of many individual bones, called vertebrae which are stacked on top of each other and formed the vertebral columns. The vertebral column has four major regions sorted from neck to pelvis. The highest region is the cervical spine. The next region, located in the upper and mid-back, is called the thoracic spine. Below the thoracic spine is the lumbar spine, in the lower back. Lastly, the sacral spine is located below the lumbar spine and connected to the pelvis. The vertebrae are connected to each other by sturdy intervertebral discs. These intervertebral discs act as cushions and shock absorbers between the vertebrae.

Various reasons such as degenerative arthritis, development of a tumor, infection, scoliosis, genetic disorders, spondylolisthesis or fracture can cause back pain and spinal pathologies for which surgical intervention may be necessary. Spinal fusion is a unique orthopedic surgical technique that permanently connects two or more adjacent vertebrae and inhibits any mobilization of the fused vertebrae while encouraging vertebrae to grow back together.

Many systems and assemblies have been disclosed in the art that provide fixation and immobilization of the adjacent vertebrae by embedding artificial implants either inside or outside of the vertebral column. These systems usually include a cage as a space holder between vertebrae and at least one insert or fastener to secure the cage to the bones.

### BRIEF SUMMARY OF THE INVENTION

The present invention is a stand-alone interbody spinal cage for fusion of adjacent vertebrae by a new technique. This device includes an anatomical spacer (cage) having a superior and an inferior surface in contact with vertebrae, and superior and inferior fixation holes that provide pathways for inserting the pins/bars/fasteners into the bone. The fixations holes can be located in any position on the superior and inferior surface. The invention includes channels passing through the spacer and are extended from opening holes to the holes on the superior and inferior surfaces of the spacer. These channels may have straight trajectory at the beginning and curved trajectory (e.g., helical, etc.) close to superior and inferior surfaces. These channels guide the pins/bars/fasteners through the spacer and give a curved shape to the pins/bars/fasteners while they are being inserted into the vertebrae.

According to one aspect of the invention, the spacer of the device may have optional cavities, extended from the superior surface to the inferior surface for holding the bone graft or autograft within the spacer depending on the surgeon's desire. This feature promotes fusion of device to the bone and increases the stability of the device.

According to another aspect of the invention, the spacer of the device has a feature (e.g., a hole on front (anterior) surface) which is engaged with an inserter for placing the spacer within the adjacent vertebrae. Moreover, this feature can be used for injecting bone graft, bone morphogenetic protein (BMP), autograft, etc. into the graft cavity.

According to another aspect of the invention, the spacer of the device can be made using a porous material/structure to allow bone ingrowth within the device and provide additional fixation.

Additionally, this device includes pins/bars/fasteners that can have any cross-section and configuration, which are passed through the spacer and inserted into the bone. Initially, the shape of the pins/bars/fasteners may be straight, but they may be formed to a curved shape after being inserted into the spacer. The other option is that the shape of the pins/bars/fasteners may be curved at beginning, but they may be formed to a straight shape after being inserted into the spacer or they can be curved or straight the entire length. The shape of the pins/bars/fasteners can be selected such that provides maximum spacer to bone fixation, stability, ease of insertion, and ease of revision. Each pin/bar/fastener may have features that mates with features on the spacer that locks the pin/bar/fastener in place and prevents extra insertion of the pins/bars/fasteners into the vertebrae or back out of pin/bar/fastener. The curved pins/bars/fasteners may have different radii in different planes (e.g., sagittal, frontal, and transverse) to maximize the fixation of the device to bone.

The pins/bars/fasteners may be configured with various extensions/sizes. For instance, one of them can have a higher protrusion from the superior surface compared to inferior surface or vice versa or even from one opening hole on the superior surface compared to one opening hole on the inferior surface.

The device may be made from any biocompatible materials. For instance, the spacer may be manufactured from a biocompatible metal, ceramic, polyether ether ketone (PEEK), etc. In the present invention, the pins/bars/fasteners may be formed from a similar or different material compared to the spacer. For example, the spacer may be made from PEEK and the pins/bars/fasteners may be made from Nitinol.

According to one aspect of the invention, the pins/bars/fasteners of the device can be made using a porous material/structure to allow bone ingrowth within the pins/bars/fasteners and provide additional fixation.

### BRIEF DESCRIPTION OF THE DRAWINGS

The invention is best understood from the following detailed description when read in connection with the associated drawings. It is emphasized that, according to common practice, the various features of the drawing are not to scale. On the contrary, the dimensions of various features are arbitrarily expanded or reduced for clarity.

Included in the drawing are the following figures.

FIG. 1A is a perspective view of embodiment appropriate for interbody vertebral fusion including spacer integrated with inserts/pins/bars to secure the device to adjacent vertebrae.

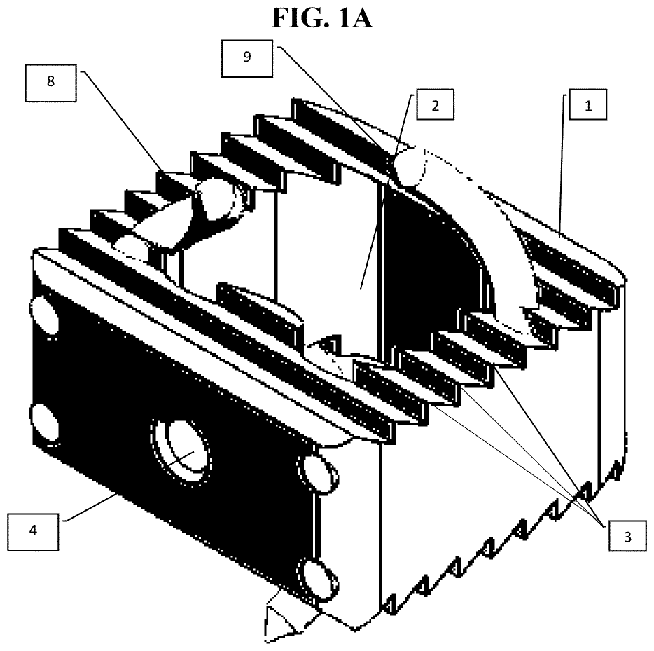

FIG. 1B is an anterior (front) view of the embodiment shown in FIG. 1A;

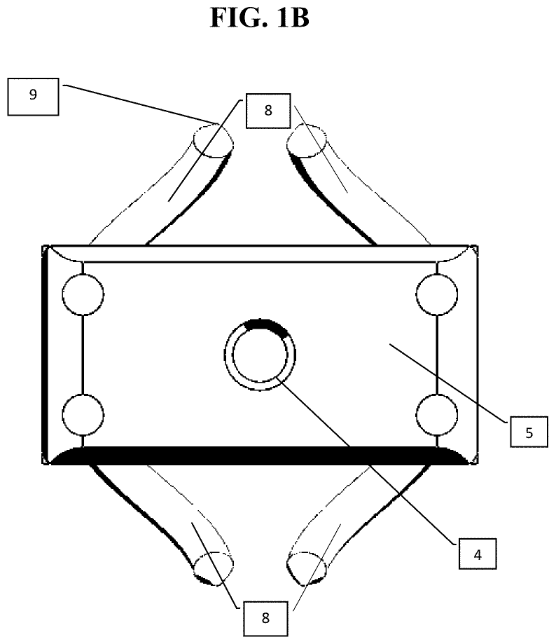

FIG. 1C is a superior (top) view of the embodiment shown in FIG. 1A;

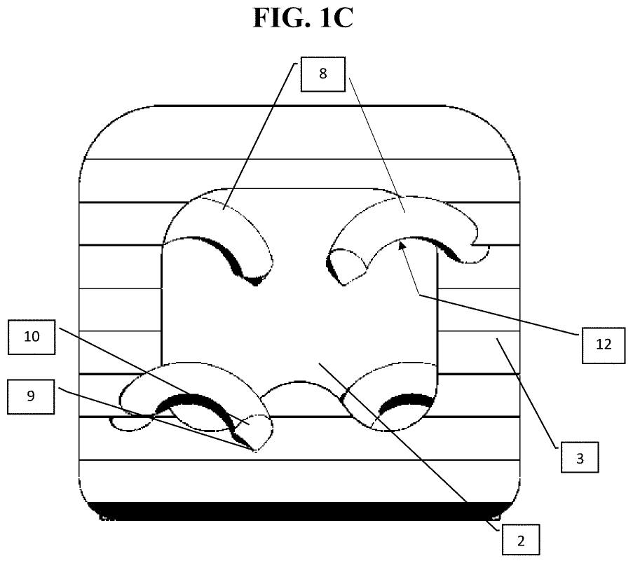

FIG. 1D is a lateral (side) view of the embodiment shown in FIG. 1A;

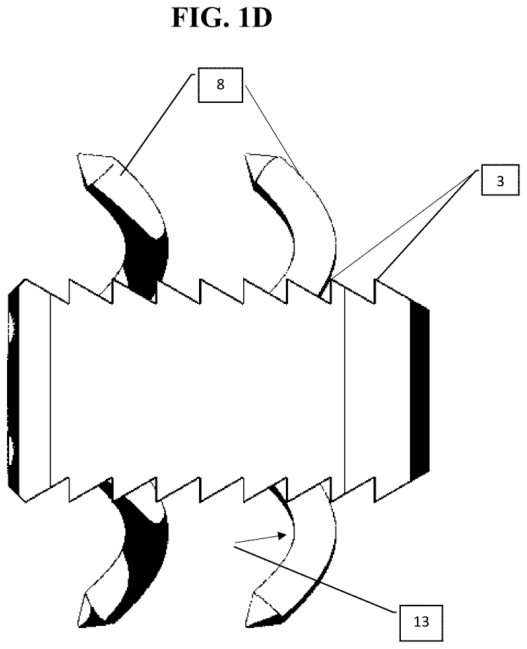

FIG. 1F is an exploded view of the embodiment shown in FIG. 1A;

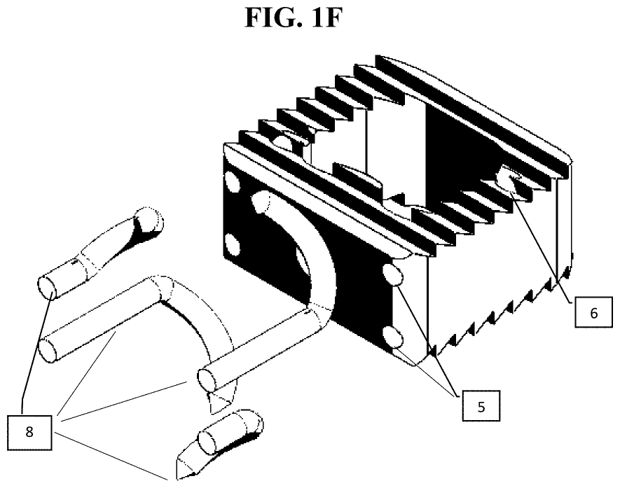

FIG. 1G is an exploded view of the embodiment shown in FIG. 1A prior pins/bars/fasteners are being inserted through the spacer;

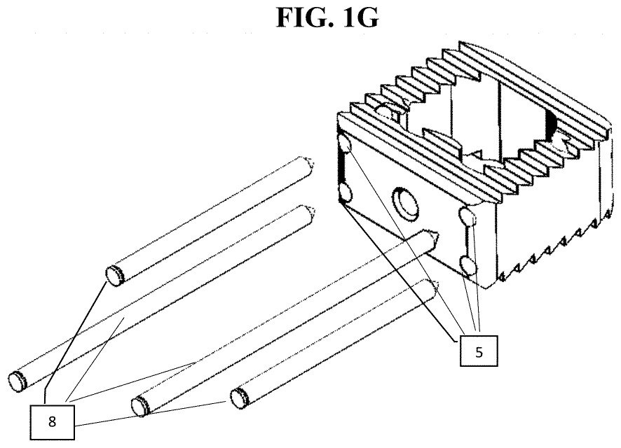

FIG. 2 is an illustration of the intervertebral device of FIG. 1A used in the spine.

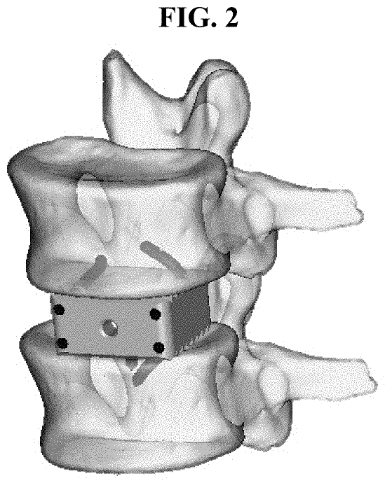

FIG. 3A is a perspective view of inserts/pins/bars prior to deploying into spacer;

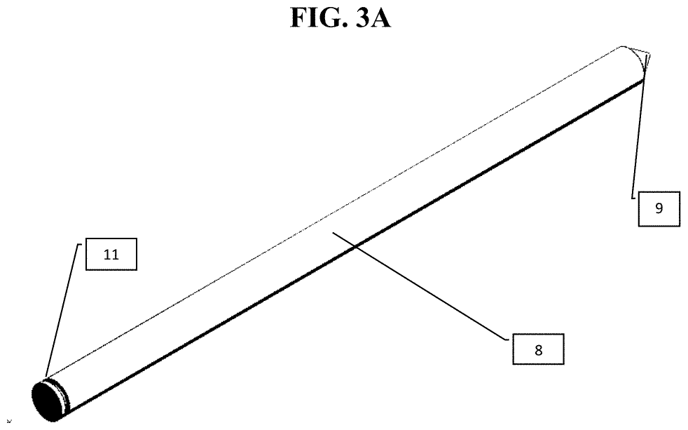

FIG. 3B is a perspective view of inserts/pins/bars after deploying into spacer;

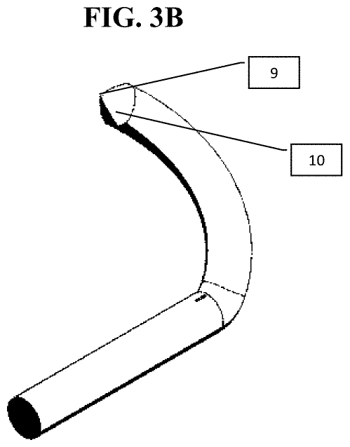

FIG. 3C is an anterior (front) of the inserts shown in FIG. 3B;

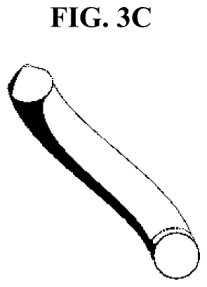

FIG. 3D is a superior (top) view of the inserts shown in FIG. 3B;

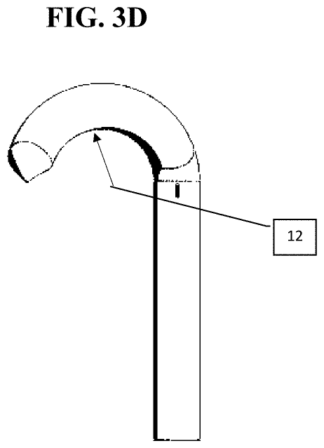

FIG. 3E is a lateral (side) view of the inserts shown in FIG. 3B;

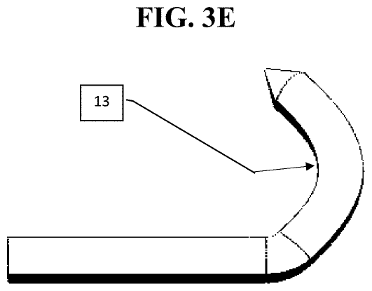

FIG. 4A is a perspective view of the spacer;

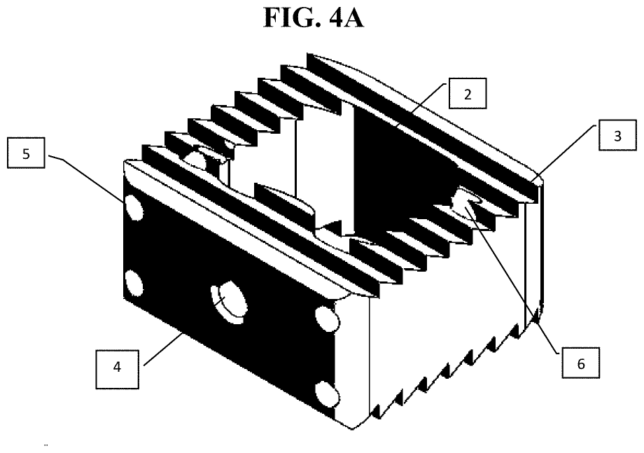

FIG. 4B is a perspective view of the spacer with increased transparency;

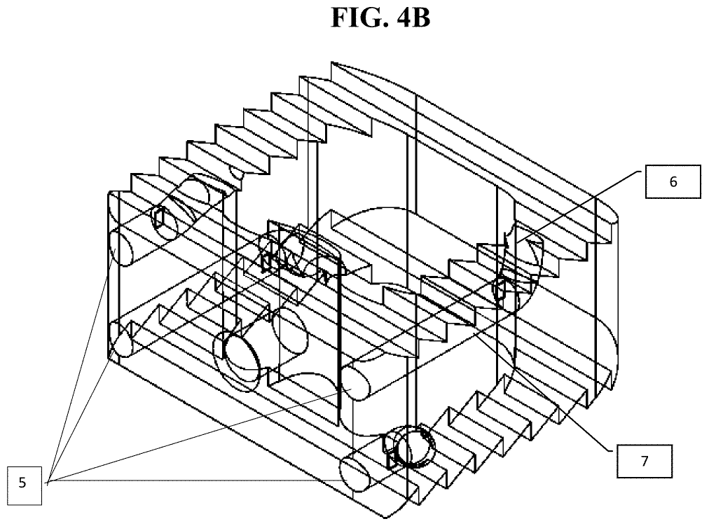

FIG. 4C is an anterior (front) of the spacer shown in FIG. 4B;

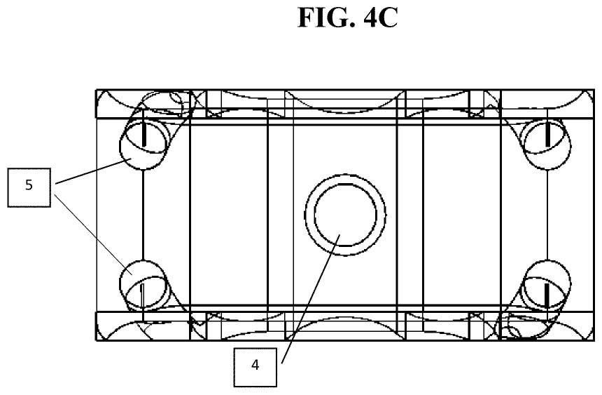

FIG. 4D is a superior (top) view of the spacer shown in FIG. 4B;

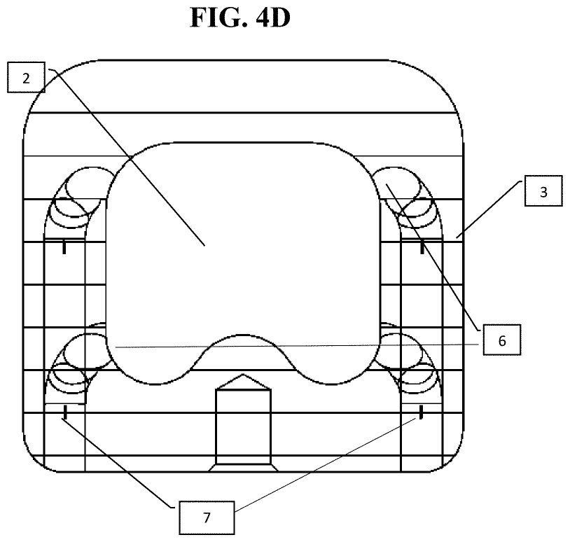

FIG. 4E is a lateral (side) view of the spacer shown in FIG. 4B;

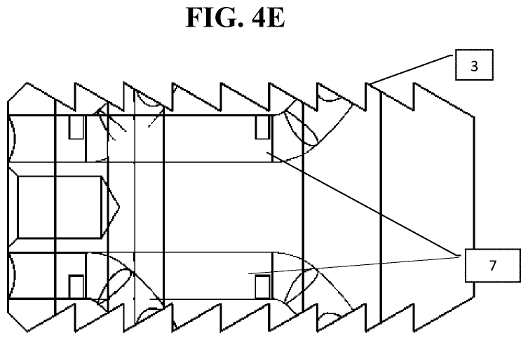

### DETAILED DESCRIPTION AND BEST MODE OF IMPLEMENTATION

The embodiments of present invention provide stand-alone interbody fusion device that may be designed or adopted in different styles for use in various levels of the spine including the cervical spine, the thoracic spine, the lumbar spine, and the sacrum spine. These various styles may be an anterior cervical discectomy and fusion (ACDF) device, a posterior cervical discectomy and fusion (PCDF), a far anterior lateral interbody fusion (FALIF) device, a posterior lumbar interbody fusion (PLIF) device, a transforaminal lumbar interbody fusion (TLIF) device, an anterior lumbar interbody fusion (ALIF) device, and a lateral lumbar interbody fusion (LLIF). Therefore, this closure applies with equal weight to every level of the spine.

The outer body of this device may have different surface features, surface coating, surface finish, and surface textures in order to provide resistance against bony parts, achieve sufficient stability, and form a bony on-growth to the implant, thereby promoting fusion.

The body of the spacer 1 may include optional cavities 2, extended from inferior surface to superior surface, for the use of containing different bone graft material such as bone marrow aspirate/concentrate, bone morphogenetic protein (BMP), autograft, etc. Additionally, the body of the spacer may be made from a variety of biocompatible materials including biocompatible implantable polymers such as PEKK, PEKEK, polyetheretherketone (PEEK), CRF (carbon reinforce peak), Titanium, Nitinol, ceramic, SI3N4, etc. The pin/bar/fastener may be made of a different biocompatible material, including but not limited to, titanium, nitinol, and stain steel, etc.

The spacer 1 and pins/bars/fasteners 8 may have different geometrical profiles, shapes, and sizes for the purpose of covering a variety of patient anatomy and spinal disorders. In the present invention, at least one pins/bars/fasteners may be deployed into the spacer in order to provide bone to implant fixation. During implantation, the pins/bars/fasteners may be inserted either sequentially or simultaneously using a designed inserter. Each pin/bar/fastener 11 may have features which mate with features within the spacer to prevent back out or progress of the pins/bars/fasteners from/in the spacer after being fully deployed. The outer profile 8 of the pins/bars/fasteners may be manufactured with various profiles such as square, ellipse, rectangle, etc. This profile will be chosen in such way that the pins/bars/fasteners withstand the physiological loading condition applied on spine, deliver maximum torsional and pull out stability, and easily being penetrated into the bone. The tip of the pins/bars/fasteners 9 may be formed with a sharply pointed shape. This feature facilitates the penetration of the pins/bars/fasteners into the cortical and trabecular bone. The cone region 10 and the outer profile 8 of pins/bars/fasteners may have geometrical features that increase the fixation of the pins/bars/fasteners into bone when the pins/bars/fasteners are implanted into the bone.

The pins/bars/fasteners can be deployed into the spacer by an inserter using different techniques, including but not limited to, impaction, driving mechanism, etc.

The spacer 1 of the present invention includes superior surfaces, an inferior surface, and a peripheral surface. The inferior and superior surfaces are in a direct contact with adjacent vertebrae and are formed with different surface features. This surface features 3 may be made with various shapes and roughness in order to achieve maximum spacer to bone fixation, stability, and bone in-growth. The spacer has channels which are extended from a designed opening hole on the peripheral surface 5 of the spacer to the opening holes on the superior and inferior surface 6 of the spacer. The opening holes on the superior and inferior surfaces can be located in any positions in order to provide desired stability and fixation. Channels 7 may have straight pathway at the beginning and curved pathway close to the superior and inferior opening holes. The other option is that these channels 7 may have curved pathway at the begging and straight pathway close to the superior and inferior opening holes or they can be curved or straight the entire pathway. The shape of the pathways can be selected such that provide maximum spacer to bone fixation, stability, ease of insertion, and ease of revision. The purpose of these channels is to guide the pins/bars/fasteners throughout the spacer. The curved region of channels may be made with different radii in different anatomical planes to form the pins/bars/fasteners with different radii in different planes 12, 13. The profile, size, shape of these channels 7 may vary based on the outer profile of designed pins/bars/fasteners. Additionally, the spacer of the device has a feature 4 (e.g., a hole on front (anterior) surface) which is engaged with the inserter for placing the spacer within the adjacent vertebrae. This feature also allows the surgeon to inject the bone graft, bone morphogenetic protein (BMP), autograft, etc. into the spacer.

Although the invention has been described in detail and reference to specific embodiments, it will be apparent to one skilled in the art that various changes and modification can be made without departing from the spirit and scope of the invention.

1. An intervertebral implant for treatment of a spine compromising:
1. A spacer placed between two adjacent vertebrae with configured channels to receive and guide the pins/bars/fasteners into the bone.
2. A pin/bar/fastener or a set of pins/bars/fasteners configured to pass through the spacer passages, follow the trajectory of the channels and penetrate into the bone.
